(19) United States
(12) Patent Application Publication
(10) Pub. No.: US 2005/0118266 A1
(43) Pub. Date: Jun. 2, 2005

(54) Sustained/controlled release solid formulation as a novel drug delivery system with reduced risk of dose dumping

(76) Inventors: M.Z.I. Khan, Zagreb (HR); Aleksandra Krajacic, Zagreb (HR); Zdravka Knezevic, Zagreb (HR); Snjezana Vodopija-Mandic, Kutina (HR)

Correspondence Address: DARBY & DARBY P.C., P. O. BOX 5257, NEW YORK, NY, 10150-5257, US

(21) Appl. No.: 10/504,014

(22) PCT Filed: Mar. 27, 2002
(86) PCT No.: PCT/HR02/00018

(30) Foreign Application Priority Data
Feb. 11, 2002 (HR) P20020124A

(51) Int. Cl. A61K 31/4172; A61K 9/22; A61K 9/26

(52) U.S. Cl. 424/470; 514/400

(57) ABSTRACT

A sustained/controlled release formulation with reduced risk of dose dumping and side effects combines two components: component (a) comprises a pharmaceutically active agent and a water-insoluble, but water-permeable polymer, whereas component (b) comprises a pharmaceutically active agent and a hydrophobic material. By changing the ratio of a pharmaceutically active agent and water-insoluble, but water-permeable polymer comprised in the component (a) and/or the ratio of the pharmaceutically active agent and hydrophobic material comprised in the component (b), and ideal release rate, with reduced risk of dose dumping and side effects, can easily be achieved.

### BACKGROUND OF THE INVENTION

1. Technical Field

Present innovation is related to a sustained/controlled release formulation for solid pharmaceuticals, primarily designed for oral administration. The innovation is referred to a two-component system which ensures sustained release of the active substance, therefore administration of a single dosage unit once or twice daily.

2. Description of the Background of the Invention

The advantages of drug delivery in a controlled manner have been described in the literature (e.g. Khan, M. Z. I, Drug Dev. Ind. Pharm., 21 (1995) 1037-1070). Most importantly, controlled/sustained release dosage forms allow the drug(s) to be released in optimum amounts, minimising unwanted side effects over a prolonged period, thus obviating the need for multiple administration. Not surprisingly, controlled/sustained release dosage forms have now become the state of the art in the area of drug delivery technology. Large number of drug delivery systems which would release a sufficient amount of drug(s) for the initial bioavailability for faster action, followed by a controlled/sustained release for prolonged/continuous action over time have been described, e.g:
international patent WO 9641617 describing tablets which comprises two layers: 1) containing a pharmaceutically active agent in a derivative, water-soluble form, combined with excipients which enable processing and immediate release of the active substance, and 2) containing the same active ingredient as the layer 1), but in a lower soluble, non-derivative form, together with excipients which enable processing and which, additionally, modify drug release; consequently the active ingredient is released from different layers at different rates
patent U.S. Pat. No. 5,164,193 (EP 468 436) describing matrix tablets which comprises two powders: A) combining an active substance, oil component and a water-insoluble polymer, and B) combining an active substance and a water-soluble polymer
patent U.S. Pat. No. 6,083,533 describing controlled release layered tablets which comprise a layer matrix with at least one cover layer lying on, characterised by thickness gradients and capability to control the release rate of the active substance(s) due to eroding in the liquid
patent U.S. Pat. No. 6,183,778 describing pharmaceutical tablets capable of liberating one or more drugs at different release rates due to comprising at least three layers, wherein the first layer controls the release rate of the drug substance(s) due to swelling or solubilizing, enabling immediate or sustained drug release, the second layer controls the release rate of the drug substance(s) due to swelling, eroding or gelling enabling sustained release of the drug substance(s) and the third layer as, at least, partial coating of one or more free surfaces of the second layer, possessing the capability to swell, erode or form gel when contacted with aqueous liquids
patent U.S. Pat. No. 6,294,199 describing a method of treating a bacterial infection by amoxycillin modified release tablets which comprise part of amoxycillin formulated with pharmaceutical excipients which allow immediate release of the drug, whereas the remaining part of amoxycillin is formulated with pharmaceutical excipients which allow slow release of the drug substance.

One of the major problems associated with controlled/sustained release dosage forms described in these and other patents and in the scientific literature in general is the possibility of dose dumping. Most of these systems do not offer a mechanism of minimising the risk of dose dumping which can seriously affect patients' safety and tolerability. The present invention offers therapeutic effect over prolonged period of time with minimised risk of dose dumping due to dual mechanism of controlling the release of the active agent from the dosage form. By changing the ratio of two components comprised in the present system, an ideal release rate with maximum relief for the patient can easily be achieved, with minimised risk of side effects and/or toxicity.

Also, most controlled/sustained release drug delivery systems require sophisticated technology, which is not available in standard facilities. In contrast, the manufacturing process and technology described in this invention involves standard technologies and equipment commonly used for manufacture of conventional dosage forms.

Biopharmaceutics Classification Scheme (BCS) categorises drug substances into four basic groups according to their solubility and capability to penetrate into plasma through the gastrointestinal wall (e.g. Dressman, J. B et al, Pharm. Res., 15(1) (1998) 11-22). Drug substances belonging to Class I are highly soluble and highly permeable. Drug substances belonging to Class II are poorly soluble and highly permeable. Drug substances belonging to Class III are highly soluble and poorly permeable, whereas substances belonging to Class IV are poorly soluble and poorly permeable drugs.

An object of the present invention is to provide an oral controlled/sustained release formulation with minimised risk of dose dumping and side effects, or, at least, to provide the public with a useful choice, independently of the solubility and permeability of the drug substances.

### SUMMARY OF THE INVENTION

Accordingly, in the first aspect the present invention provides a solid controlled release oral dosage formulation, comprising two components wherein:
a) the first component comprises granules containing a pharmaceutically active agent intermixed with a pharmaceutically acceptable, water-insoluble, water-permeable polymeric material; and
b) the second component comprises a mixture of a pharmaceutically active agent and at least 2% (w/w) of a water-insoluble, water-impermeable hydrophobic material.

In another aspect, the present invention provides a method of preparation of a sustained release solid dosage form including two components wherein:
a) the first component comprises granules containing a pharmaceutically active agent intermixed with a pharmaceutically acceptable, water-insoluble, water-permeable polymeric material; and
b) the second component comprises a mixture of a pharmaceutically active agent and at least 2% (w/w) of a water-insoluble, water-impermeable hydrophobic material.

In a further aspect, the present invention provides the use of a sustained release solid dosage formulation including two components wherein:
a) the first component comprises granules containing a pharmaceutically active agent intermixed with a pharmaceutically acceptable, water-insoluble, water-permeable polymeric material; and
b) the second component comprises a mixture of a pharmaceutically active agent and at least 2% (w/w) of a water-insoluble, water-impermeable hydrophobic material.

In a still further aspect, the present invention provides a method of minimising dose dumping comprising administering to a patient in need thereof a sustained release solid dosage formulation including two components wherein:
a) the first component comprises granules containing a pharmaceutically active agent intermixed with a pharmaceutically acceptable, water-insoluble, water-permeable polymeric material; and
b) the second component comprises a mixture of a pharmaceutically active agent and at least 2% (w/w) of a water-insoluble, water-impermeable hydrophobic material.

In a yet further aspect, the present invention provides a use, in the preparation of a sustained release solid dosage form for a sustained release of a pharmaceutically active agent in a patient in need thereof, of:
a) granules containing a pharmaceutically active agent intermixed with a pharmaceutically acceptable, water-insoluble, water-permeable polymeric material; and
b) a pharmaceutically active agent and at least 2% (w/w) of a water-insoluble, water-impermeable hydrophobic material.

In another aspect, the present invention provides a process for the production of a sustained release solid dosage formulation, including combining:
a first component comprising a granules, containing a pharmaceutically active agent intermixed with a pharmaceutically acceptable water-insoluble, but water-permeable polymeric material; with
a second component comprising a mixture of a pharmaceutically active agent and at least 2% (w/w) of a water-insoluble, water-impermeable hydrophobic material.

Although the invention is broadly defined above, it is not limited thereto and, also, includes embodiments of which the following description provides examples.

### BRIEF DESCRIPTION OF THE DRAWINGS

FIG. 1 is a graph depicting sustained release using a dosage formulation of the invention. It depicts controlling/sustaining the release rate by changing the amount of water-insoluble (but water-permeable) polymeric material. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, diclofenac sodium, contained in the dosage form is poorly soluble and highly permeable (Class II, according to Biopharmaceutics Classification System).

FIG. 2 is a graph depicting sustained release using a dosage formulation of the invention. It depicts controlling/sustaining the release rate by changing the amount of lipid/lipidic component as an additional retarding agent. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, diclofenac sodium, contained in the dosage form is poorly soluble and highly permeable (Class II, according to Biopharmaceutics Classification System).

FIG. 3 is a graph depicting sustained release using a dosage formulation of the invention. It depicts controlling/sustaining the release rate by changing the amount of water-insoluble (but water-permeable) polymeric material and/or lipid/lipidic component. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, diclofenac sodium, contained in the dosage form is poorly soluble and highly permeable (Class II, according to Biopharmaceutics Classification System).

FIG. 4 is a graph depicting sustained release using a dosage formulation of the invention. It depicts influence of the water-soluble excipients in granules and/or water-insoluble excipients in the tablet blend. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, diclofenac sodium, contained in the dosage form is poorly soluble and highly permeable (Class II, according to Biopharmaceutics Classification System).

FIG. 5 is a graph depicting sustained release using a dosage formulation of the invention. It depicts controlling/sustaining the release rate by changing the amount of water-insoluble (but water-permeable) polymeric material. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, torasemide, contained in the dosage form is highly soluble and highly permeable (Class I, according to Biopharmaceutics Classification System).

FIG. 6 is a graph depicting sustained release using a dosage formulation of the invention. It depicts controlling/sustaining the release rate by changing the amount of lipid/lipidic component as an additional retarding agent. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, torasemide, contained in the dosage form is highly soluble and highly permeable (Class I, according to Biopharmaceutics Classification System).

FIG. 7 is a graph depicting sustained release using a dosage formulation of the invention. It depicts controlling/sustaining the release rate by changing the amount of water-insoluble (but water-permeable) polymeric material and/or lipid/lipidic component.

Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, torasemide, contained in the dosage form is highly soluble and highly permeable (Class I, according to Biopharmaceutics Classification System).

FIG. 8 is a graph depicting sustained release using a dosage formulation of the invention. It depicts controlling/sustaining the release rate by changing the amount of water-insoluble (but water-permeable) polymeric material. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, ranitidine (in the form of ranitidine hydrochloride), contained in the dosage form is highly soluble and poorly permeable (Class III, according to Biopharmaceutics Classification System).

FIG. 9 is a graph depicting sustained release using a dosage formulation of the invention. It depicts controlling/sustaining the release rate by changing the amount of lipid/lipidic component as an additional retarding agent. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, ranitidine (in the form of ranitidine hydrochloride), contained in the dosage form is highly soluble and poorly permeable (Class III, according to Biopharmaceutics Classification System).

FIG. 10 is a graph depicting sustained release using a dosage formulation of the invention. It depicts controlling/sustaining the release rate by changing the amount of water-insoluble (but water-permeable) polymeric material and/or lipid/lipidic component. Dissolution testing was carried out by using USP apparatus I at 150 rpm. For the first 30 minutes the release profile was tested in diluted hydrochloric acid (pH 2.5) and then in mixed phosphate buffer (pH 6.8) for 8 hours. The data represent mean values obtained from 6 tablets. The pharmaceutically active agent, ranitidine (in the form of ranitidine hydrochloride), contained in the dosage form is highly soluble and poorly permeable (Class III, according to Biopharmaceutics Classification System).

### DETAILED DESCRIPTION OF THE INVENTION

As defined above, the present invention relates to a novel solid sustained/controlled release oral dosage formulation.

The formulation of the invention comprises a two-component system. The first component comprises an active pharmaceutical agent in combination with a water-insoluble, but water-permeable polymer.

The first component is preferably in the form of granules and is capable of sustaining the release of the active agent over a prolonged period of time, depending on the amount of polymer, either if the shape of the dosage form remains intact or even if it is disintegrated into small pieces.

Preferably, the water-insoluble, but water-permeable polymeric material comprises one or more methacrylic acid copolymers, ethylcellulose or mixture thereof and others with similar properties. Conveniently, the water-insoluble, but water-permeable polymeric material is presented in an amount within the range of from about 2-90% (w/w) and/or in the proportion to the active substance from (1:10) to (10:1).

The second component of the system contains an active pharmaceutical agent, untreated with the water-insoluble polymer, and available for substantially immediate release, depending on the physico-chemical properties of the active agent.

The second component of the formulation also contains a hydrophobic, preferably a lipid or lipidic material. More preferably, the hydrophobic material is selected from the group of glycerine fatty acid esters, vegetable oils and their derivatives, higher fatty acids, their metal salts and other material with similar properties. It will be appreciated by art-skilled workers that the release rate of the active agent in the second component is controlled by the amount of the hydrophobic material presented in the formulation in an amount within the range of from about 2-80% (w/w) and/or in the proportion to the active substance from (1:10) to (10:1). The second component is conveniently not granulated, but can, also, be in the form of granules in which the hydrophobic material (e.g. lipid) can be added in melted state, if required.

Without wishing to be bound by theory, it is believed that the risk of dose dumping in the present invention is minimised due to dual mechanism of controlling/sustaining the release process due to the dual component system. In the case of an oral dosage formulation of the invention, the main role of the hydrophobic (second) component of the system is to control the penetration rate of the gastrointestinal fluid into the dosage form and, thereby, to control the release of the drug available in untreated form (in the second component). As a result of a control of gastrointestinal fluid penetration rate, the hydrophobic component also, indirectly, controls the release of the drug available inside the granules. In other words, the release of the drug available in the granules (first component) is controlled by both the water-insoluble, but water-permeable polymer and, also, the hydrophobic material in the second component. Therefore, if the system (the dosage form) fails accidentally (e.g. as a result of food intake) or naturally (due to gastrointestinal motility), the risk of dose dumping is minimised because the first component would not release the drug due to control by the water-insoluble, but water-permeable polymer.

Preferably, the pharmaceutically active agent of the second component is the same as that of the first component. The pharmaceutically active agent may also comprise a mixture of agents. Having the same active pharmaceutical agent in the first and second components affords a formulation in which part of the active agent is available for substantially immediate release (depending on the quantity of hydrophobic material added), and part of the active agent will be released over a prolonged period of time. However, formulations in which the first and second components comprise different pharmaceutically active agents are also contemplated and are by no means excluded.

The first component may be prepared by combining the pharmaceutically active agent with a polymeric substance that is insoluble in water, but permeable to water. As a result, the release rate of the active agent from the first component can be controlled by adjusting the amount of the polymer, depending on the physico-chemical characteristics of the active agent. In addition, standard pharmaceutical excipients can be used to obtain granules with appropriate compressibility for tabletting.

Preferably, the first component is in granular form and two components are in the admixture.

Optionally, the first component may also contain one or more pharmaceutically acceptable excipients. Examples of suitable excipients include (but are not limited to) lactose and/or microcrystalline cellulose, croscarmellose sodium, starch and/or starch derivatives. Such excipients can also be used to enhance the permeability of water to the granules, and, consequently, enhance the release rate of the drug if required. Lactose and microcrystalline cellulose are examples of suitable filler excipients.

Generally, the second component of the system contains the pharmaceutically active agent available for substantially immediate release. However, the release process can be controlled by the amount of hydrophobic material in the second component.

Optionally, the second component may also contain one or more pharmaceutically acceptable excipients and/or tabletting aids. Fillers, glidants, lubricants and mixtures thereof may also be provided in the second component. Non-limiting examples include calcium hydrogen phosphate and hydrogenated vegetable oil NF, Type I. Conveniently, these may be mixed with talc and magnesium stearate.

Preferably, the dosage form is a tablet or capsule. In a particularly preferred embodiment, the dosage formulation is an oral dosage formulation. However, sustained release profiles afforded by a dosage formulation of the invention make it suitable to be adapted to many different types of dosage forms. Non-limiting examples of other dosage forms contemplated include suppositories and subcutaneous implants.

Controlled release oral dosage formulations of the invention may be in the form of tablet compressed from a blend of the two components, and, also:
(i) hard capsules (such as gelatin capsules) filled with a mixture of the first and second components
(ii) hard (e.g. gelatin) capsules filled with one or more compressed tablets comprising the first and second components, or
(iii) hard (e.g. gelatin) capsules containing both granules (the first component) or mixture of the second component and one or more tablets.

If the desired pharmaceutical dosage form is a tablet, the granules (the first component) are mixed with the second component which comprises: the active agent, a hydrophobic material (preferably a lipid or lipidic material such as fatty acids or their esters) and some tabletting materials (e.g. antiadherents, glidants, lubricants), and then compressed into tablets.

A film coating may optionally be added to the dosage formulation. The coating layer can be either non-functional (for example to give an elegant appearance, identification or colour) or functional, such as enteric coating, or to incorporate the active in the coating layer for rapid release for immediate action (instant release). The film coating may conveniently comprise one or more film formers, plasticisers, colouring agents, and mixture thereof.

The water insoluble polymeric substances suitable for granulation and/or control of the release of the drug from the granules can be chosen from, but not restricted to, the range of methacrylic acid copolymers, such as Eudragit RS or Eudragit RL (either in the form of a powder or aqueous suspension or a combination of both forms), Eudragit NE 40D or Eudragit NE 30D, or a combination of both polymers in appropriate amounts and forms (powder/suspension).

The pharmaceutically active agent is generally an agent required to be administered by sustained release. Examples of such agents include agents with toxicity in high doses and agents to be administered over an extended period of time.

The controlled release formulation of the present invention may contain active agent(s) from a variety of therapeutically active groups, such as, for example, ace-inhibitors, alkaloids, antacids, analgesics, anabolic agents, anti-anginal drugs, anti-allergy agents, anti-arrhytmia agents, antiasthmatics, antibiotics, anticholesterlolemics, anticonvulsants, anticoagulants, anti-emetics, antihistamines, antihypertensives, anti-infectives, nonsteroidal anti-inflammatory drugs (NSAIDs), steroidal anti-inflammatory drugs, central nervous system (CNS) stimulators, CNS depressants, antimigraine agents, contraceptives, cough suppresants, deodorants, dermatological agents, diuretics, fungicides, gastro-intestinal agents, vitamins, minerals polypeptides, prostaglandins, respiratory stimulans, uterine relaxants, and many others already known, as well as the new drugs.

In the case of drugs that are, according to Biopharmaceutics Classification System (BCS), highly soluble such as, for example, torasemide, venlafaxine in the form of venlafaxine hydrochloride or other salts, gabapentin, pravastatin sodium, ranitidine in the form of ranitidine hydrochloride or other salts, and others, well known and new drugs, the active ingredient contained in the second component (non-granulated form) is preferably in untreated form as a pure substance. However, in the case of drugs that are, according to BSC, poorly soluble, such as, for example, temazepam, diazepam, oxazepam, nifedipine, ibuprofen, loratadine, and others, well known and new drugs, the active ingredient contained in the second component (non-granulated form) is either in untreated form as a pure substance, or, optionally, in the form of solid dispersion in a carrier. Furthermore, the substance may be blended with pharmaceutical excipients suitable for further processing (tabletting or capsuling).

The carrier of the solid dispersion may be selected from a wide range of polymers (e.g. various types of polyethylene glycols) or other standard pharmaceutical excipients, such as, for example, polyvinyl pyrrolidone (povidones, Kollidon VA 64) and others.

In addition, solubility enhancers, such as substances capable of creating a microenvironment with optimum pH solubilization of the drug, can be included in the second component. The qualities and quantities of excipients can be determined on the basis of in-vitro experiments according to the desired release profile(s) of the drug(s).

The hydrophobic material used to control the release process from the second component (non-granulated form) is preferably chosen from a range of lipids or lipidic material, such as hydrogenated vegetable oils, pharmaceutical fats, fatty acids, glycerides, waxes and others.

The release kinetics of the active agent from the dosage formulation useful in the present invention may be effected by dual mechanism of action:
(i) from the first component by adjusting the amount of the water insoluble polymer; and
(ii) from the second component by adjusting the amount of the hydrophobic material.

Without wishing to be bound by theory, having two completely different microenvironments with two completely different release retardant mechanisms provides a very effective mechanism for controlling the overall release of pharmaceutically active agent from the formulation.

Furthermore, adjusting the proportion of the active agent in the first and second components can control the release of the active agent.

The invention will now be described in more details with reference to the following non-limiting examples.

### EXAMPLES 1-4

Tablets containing part of diclofenac sodium in granulated form using a methacrylic acid copolymer as the binder, and the remaining diclofenac sodium in non-granulated form mixed with a lipid.

### Example 1

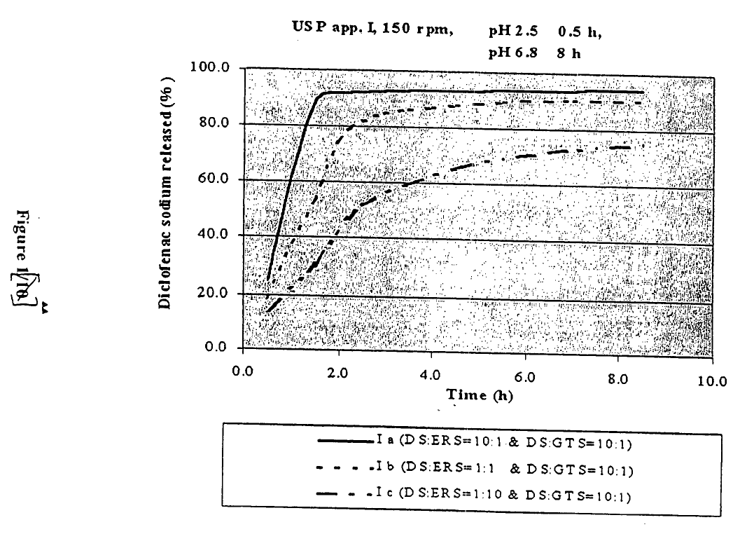

### Controlling the Release Rate by Varying the Amount of Water-Insoluble (but Water-Permeable) Polymeric Material (FIG. 1)

| Formulations | I a | I b | I c |
|---|---|---|---|
| GRANULES: Diclofenac | 10:1 | 1:1 | 1:10 |
| sodium:Eudragit RS | | | |
| Diclofenac sodium:glyceril tristearate | 10:1 | 10:1 | 10:1 |
| | I a | I b | I c |
| Composition of the tablets | (mg/tbl) | (mg/tbl) | (mg/tbl) |
| Diclofenac sodium (in granules) | 50.00 | 50.00 | 5.00 |
| Microcrystalline cellulose | 12.50 | 25.00 | 12.50 |
| Lactose | 12.50 | 25.00 | 12.50 |
| Eudragit RS | 5.00 | 50.00 | 50.00 |
| Diclofenac sodium (remaining part) | 50.00 | 50.00 | 95.00 |
| Glyceril tristearate | 5.00 | 5.00 | 9.50 |
| Hydrogenated vegetable oil NF, Type I | 5.00 | 5.00 | 5.00 |
| Calcium hydrogen phosphate (dibasic) | 10.00 | 10.00 | 10.00 |
| Talc | 5.00 | 5.00 | 5.00 |
| Magnesium stearate | 5.00 | 5.00 | 5.00 |
| Film coating | 5.00 | 5.00 | 5.00 |

Preparation of Granules Which Constitute the Continued Prolonged/Delayed Release Portion of the Tablets

Granules were prepared from a mixture of diclofenac sodium with microcrystalline cellulose, lactose and Eudragit RS as a binder, used in powder form and/or in the form of an aqueous suspension. Wet granules were dried in a fluid-bed dryer and then milled through a 20 mesh (0.8 mm) screen to obtain appropriate size distribution of the granules suitable for compressing.

Preparation of the Finished Tablets

Granules and the remaining part of the active drug, diclofenac sodium, lipid component, calcium hydrogenphosphate, hydrogenated vegetable oil NF Type I and talc were screened through a 20 mesh sieve and tumble mixed for 5 minutes. Magnesium stearate, screened through a 30 mesh (0.6 mm) sieve was then added to the final blend and mixed for another 5 minutes. The final blend was compressed into tablets. Tablets were coated with the aqueous suspension of methylhydroxypropylcellulose, polysorbatum, sodium lauryl sulfate, talc and pigments such as titanium dioxide as well as iron oxides red and yellow.

### Example 2

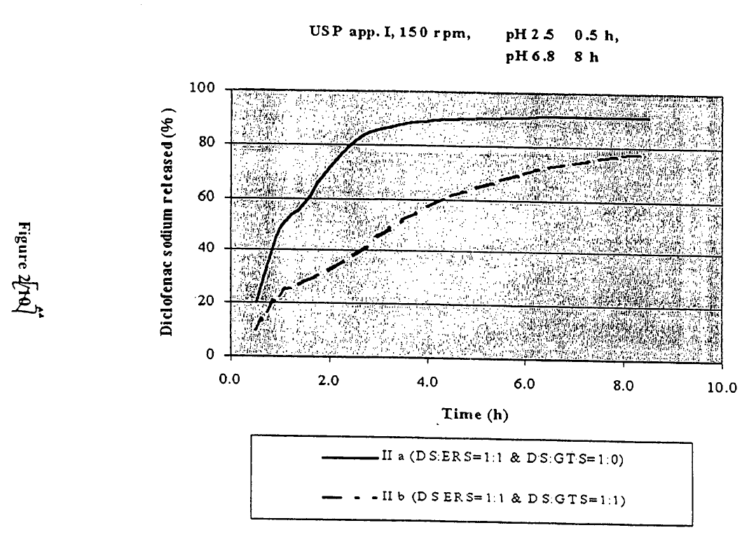

### Controlling the Release Rate by Varying the Amount of Lipid/Lipidic Component as an Additional Retarding Agent (FIG. 2)

| Formulations | II a | II b |
|---|---|---|
| GRANULES: Diclofenac sodium:Eudragit RS | 1:1 | 1:1 |
| Diclofenac sodium:glyceril tristearate | 1:0 | 1:1 |
| | II a | II b |
| Composition of the tablets | (mg/tbl) | (mg/tbl) |
| Diclofenac sodium (in granules) | 50.00 | 50.00 |
| Microcrystalline cellulose | 25.00 | 25.00 |
| Lactose | 25.00 | 25.00 |
| Eudragit RS | 50.00 | 50.00 |
| Diclofenac sodium (remaining part) | 50.00 | 50.00 |
| Glyceril tristearate | / | 50.00 |
| Hydrogenated vegetable oil NF, Type I | 5.00 | 5.00 |
| Calcium hydrogen phosphate (dibasic) | 10.00 | 10.00 |
| Talc | 5.00 | 5.00 |
| Magnesium stearate | 5.00 | 5.00 |
| Film coating | 5.00 | 5.00 |

Granules and tablets were prepared in the same way as described in the Example 1.

### Example 3

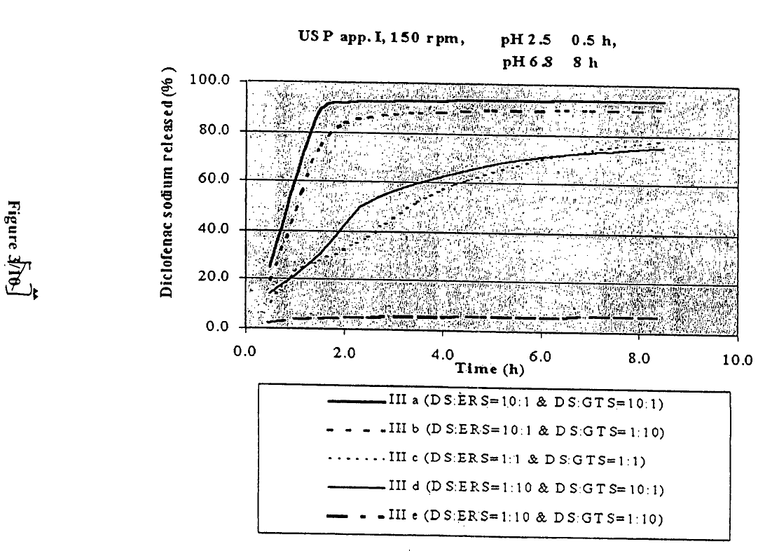

### Controlling the Release Rate by Varying Both the Amount of Water-Insoluble (but Water-Permeable) Polymeric Material and Lipid/Lipidic Component (FIG. 3)

| Formulations | III a | III b | III c | III d | III e |
|---|---|---|---|---|---|
| GRANULES: Diclofenac sodium:Eudragit RS | 10:1 | 10:1 | 1:1 | 1:10 | 1:10 |
| Diclofenac sodium:glyceril tristearate | 10:1 | 1:10 | 1:1 | 10:1 | 1:10 |
| | III a | III b | III c | III d | III e |
| Composition of the tablets | (mg/tbl) | (mg/tbl) | (mg/tbl) | (mg/tbl) | (mg/tbl) |
| Diclofenac sodium (in granules) | 50.00 | 95.00 | 50.00 | 5.00 | 10.00 |
| Microcrystalline cellulose | 12.50 | 23.75 | 25.00 | 12.50 | 25.00 |
| Lactose | 12.50 | 23.75 | 25.00 | 12.50 | 25.00 |
| Eudragit RS | 5.00 | 9.50 | 50.00 | 50.00 | 100.00 |
| Diclofenac sodium (remaining part) | 50.00 | 5.00 | 50.00 | 95.00 | 90.00 |
| Glyceril tristearate | 5.00 | 50.00 | 50.00 | 9.50 | 900.00 |
| Hydrogenated vegetable oil NF, Type I | 5.00 | 5.00 | 5.00 | 5.00 | 5.00 |
| Calcium hydrogen phosphate (dibasic) | 10.00 | 10.00 | 10.00 | 10.00 | 10.00 |
| Talc | 5.00 | 5.00 | 5.00 | 5.00 | 5.00 |
| Magnesium stearate | 5.00 | 5.00 | 5.00 | 5.00 | 5.00 |
| Film coating | 5.00 | 5.00 | 5.00 | 5.00 | 5.00 |

Granules and tablets were prepared in the same way as described in the Example 1.

### Example 4

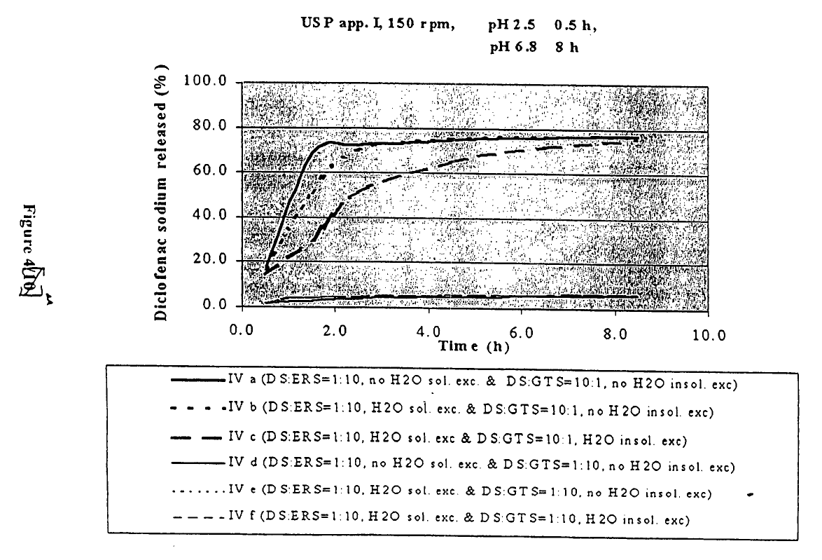

### Influence of the Excipients Either in Granules or in the Tablet Blend (FIG. 4)

| Formulations | IV a | IV b | IV c | IV d | IV e | IV f |
|---|---|---|---|---|---|---|
| GRANULES: Diclofenac sodium:Eudragit RS | 1:10 | 1:10 | 1:10 | 1:10 | 1:10 | 1:10 |
| Lactose + microcrystalline cellulose in granules | / | + | + | / | + | + |
| Diclofenac sodium:glyceril tristearate | 10:1 | 10:1 | 10:1 | 1:10 | 1:10 | 1:10 |
| Hydrogenated vegetable oil NF, Type I + Calcium | / | / | + | / | / | + |
| hydrogen phosphate (dibasic) in | | | | | | |
| tablet blend | | | | | | |
| | IV a | IV b | IV c | IV d | IV e | IV f |
| Composition of the tablets | (mg/tbl) | (mg/tbl) | (mg/tbl) | (mg/tbl) | (mg/tbl) | (mg/tbl) |
| Diclofenac sodium (in granules) | 5.00 | 5.00 | 5.00 | 10.00 | 10.00 | 10.00 |
| Microcrystalline cellulose | / | 12.50 | 12.50 | / | 25.00 | 25.00 |
| Lactose | / | 12.50 | 12.50 | / | 25.00 | 25.00 |
| Eudragit RS | 50.00 | 50.00 | 50.00 | 100.00 | 100.00 | 100.00 |
| Diclofenac sodium (remaining part) | 95.00 | 95.00 | 95.00 | 90.00 | 90.00 | 90.00 |
| Glyceril tristearate | 9.50 | 9.50 | 9.50 | 900.00 | 900.00 | 900.00 |
| Hydrogenated vegetable oil NF, Type I | / | / | 5.00 | / | / | 5.00 |
| Calcium hydrogen phosphate (dibasic) | / | / | 10.00 | / | / | 10.00 |
| Talc | 5.00 | 5.00 | 5.00 | 5.00 | 5.00 | 5.00 |
| Magnesium stearate | 5.00 | 5.00 | 5.00 | 5.00 | 5.00 | 5.00 |
| Film coating | 5.00 | 5.00 | 5.00 | 5.00 | 5.00 | 5.00 |

Preparation of Granules Which Constitute the Continued Prolonged/Delayed Release Portion of the Tablets

Granules were prepared from a mixture of diclofenac sodium with or without microcrystalline cellulose and lactose, with Eudragit RS as a binder, used in powder form and/or in the form of an aqueous suspension. Wet granules were dried in a fluid-bed dryer and then milled through a 20 mesh (0.8 mm) screen to obtain appropriate size distribution of the granules suitable for compressing.

Preparation of the Finished Tablets

Granules and the remaining part of the active drug, diclofenac sodium, lipid component, with or without calcium hydrogenphosphate and hydrogenated vegetable oil NF Type I, and with talc were screened through a 20 mesh sieve and tumble mixed for 5 minutes. Magnesium stearate, screened through a 30 mesh (0.6 mm) sieve was then added to the final blend and mixed for another 5 minutes. The final blend was compressed into tablets. Tablets were coated with the aqueous suspension of methylhydroxypropylcellulose, polysorbatum, sodium lauryl sulfate, talc and pigments such as titanium dioxide as well as iron oxides red and yellow.

### EXAMPLES 5-7

Tablets containing part of torasemide in granulated form using a methacrylic acid copolymer as the binder, and the remaining torasemide in non-granulated form mixed with a lipid.

### Example 5

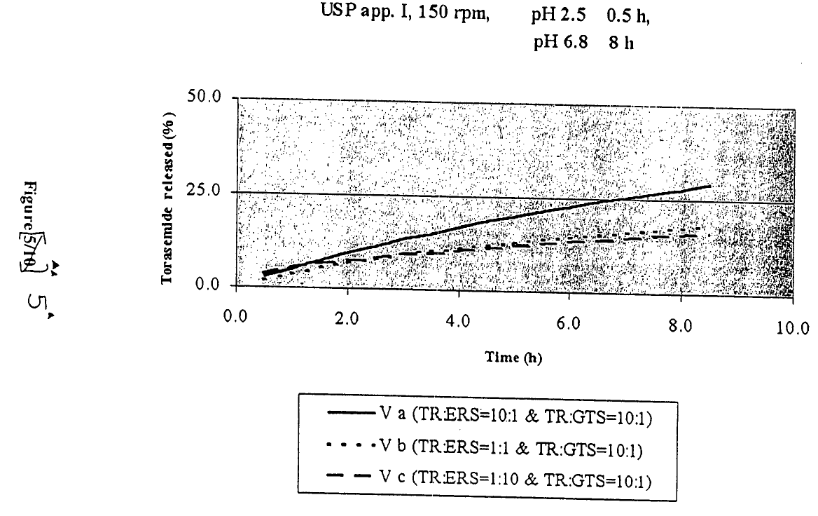

### Controlling the Release Rate by Varying the Amount of Water-Insoluble (but Water-Permeable) Polymeric Material (FIG. 5)

| Formulations | V a | V b | V c |
|---|---|---|---|
| GRANULES: Torasemide:Eudragit RS | 10:1 | 1:1 | 1:10 |
| Torasemide:glyceril tristearate | 10:1 | 10:1 | 10:1 |
| | V a | V b | V c |
| Composition of the tablets | (mg/tbl) | (mg/tbl) | (mg/tbl) |
| Torasemide (in granules) | 25.00 | 25.00 | 5.00 |
| Eudragit RS | 2.50 | 25.00 | 50.00 |
| Torasemide (remaining part) | 25.00 | 25.00 | 45.00 |
| Glyceril tristearate | 2.50 | 2.50 | 4.50 |
| Talc | 2.50 | 2.50 | 2.50 |
| Magnesium stearate | 2.50 | 2.50 | 2.50 |

Preparation of Granules Which Constitute the Continued Prolonged/Delayed Release Portion of the Tablets

Granules were prepared from a mixture of torasemide with Eudragit RS as a binder, used in powder form and/or in the form of an aqueous suspension. Wet granules were dried in a fluid-bed dryer and then milled through a 20 mesh (0.8 mm) screen to obtain appropriate size distribution of the granules suitable for compressing.

Preparation of the Finished Tablets

Granules and the remaining part of the active drug, torasemide, lipid component and talc were screened through a 20 mesh sieve and tumble mixed for 5 minutes. Magnesium stearate, screened through a 30 mesh (0.6 mm) sieve was then added to the final blend and mixed for another 5 minutes. The final blend was compressed into tablets.

### Example 6

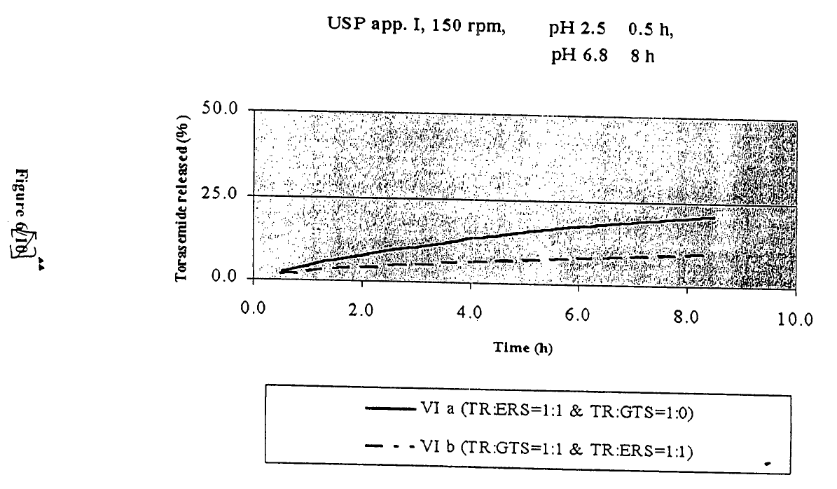

### Controlling the Release Rate by Varying the Amount of Lipid/Lipidic Component as an Additional Retarding Agent (FIG. 6)

| Formulations | VI a | VI b |
|---|---|---|
| GRANULES: Torasemide:Eudragit RS | 1:1 | 1:1 |
| Torasemide:glyceril tristearate | 1:0 | 1:1 |
| | VI a | VI b |
| Composition of the tablets | (mg/tbl) | (mg/tbl) |
| Torasemide (in granules) | 25.00 | 20.00 |
| Eudragit RS | 25.00 | 20.00 |
| Torasemide (remaining part) | 25.00 | 30.00 |
| Glyceril tristearate | / | 30.00 |
| Talc | 2.50 | 2.50 |
| Magnesium stearate | 2.50 | 2.50 |

Granules and tablets were prepared in the same way as described in the Example 5.

### Example 7

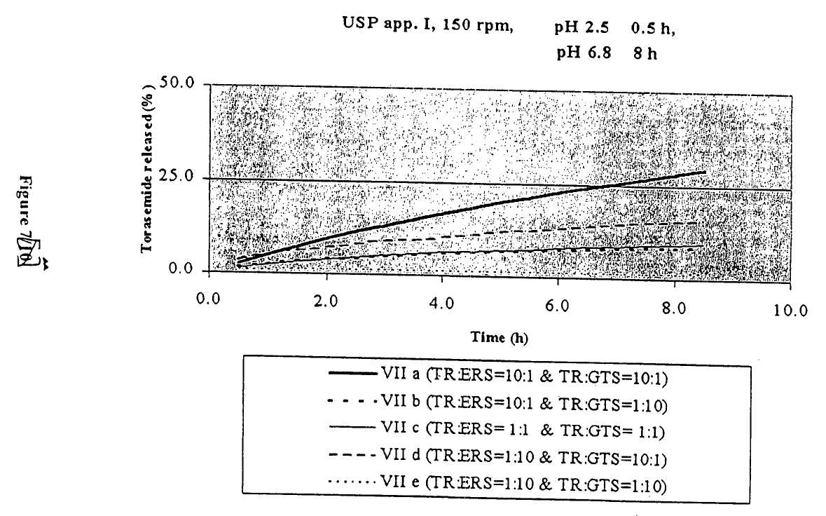

### Controlling the Release Rate by Varying Both the Amount of Water-Insoluble (but Water-Permeable) Polymeric Material and Lipid/Lipidic Component (FIG. 7)

| Formulations | VII a | VII b | VII c | VII d | VII e |
|---|---|---|---|---|---|
| GRANULES: Torasemide:Eudragit RS | 10:1 | 10:1 | 1:1 | 1:10 | 1:10 |
| Torasemide:glyceril tristearate | 10:1 | 1:10 | 1:1 | 10:1 | 1:10 |
| | VII a | VII b | VII c | VII d | VII e |
| Composition of the tablets | (mg/tbl) | (mg/tbl) | (mg/tbl) | (mg/tbl) | (mg/tbl) |
| Torasemide (in granules) | 25.00 | 45.00 | 20.00 | 35.00 | 5.00 |
| Eudragit RS | 2.50 | 4.50 | 20.00 | 350.00 | 50.00 |
| Torasemide (remaining part) | 25.00 | 5.00 | 30.00 | 15.00 | 45.00 |
| Glyceril tristearate | 2.50 | 50.00 | 30.00 | 1.50 | 450.00 |
| Talc | 2.50 | 2.50 | 2.50 | 2.50 | 2.50 |
| Magnesium stearate | 2.50 | 2.50 | 2.50 | 2.50 | 2.50 |

Granules and tablets were prepared in the same way as described in the Example 5.

### EXAMPLES 8-10

Tablets containing part of ranitidine in the form of ranitidine hydrochloride in granulated form using a methacrylic acid copolymer as the binder, and the remaining ranitidine in the form of ranitidine hydrochloride in non-granulated form mixed with a lipid.

### Example 8

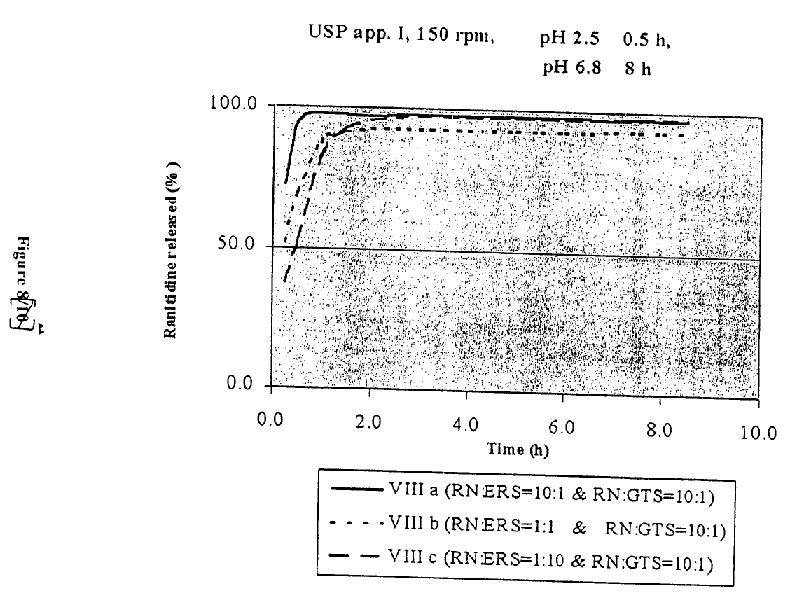

### Controlling the Release Rate by Varying the Amount of Water-Insoluble (but Water-Permeable) Polymeric Material (FIG. 8)

| Formulations | VIII a | VIII b | VIII c |
|---|---|---|---|
| GRANULES: Ranitidine:Eudragit RS | 10:1 | 1:1 | 1:10 |
| Ranitidine:glyceril tristearate | 10:1 | 10:1 | 10:1 |
| | VIII a | VIII b | VIII c |
| Composition of the tablets | (mg/tbl) | (mg/tbl) | (mg/tbl) |
| Ranitidine (in granules) | 25.00 | 25.00 | 5.00 |
| Eudragit RS | 2.50 | 25.00 | 50.00 |
| Ranitidine (remaining part) | 25.00 | 25.00 | 45.00 |
| Glyceril tristearate | 2.50 | 2.50 | 4.50 |
| Hydrogenated vegetable oil NF, Type I | 2.50 | 2.50 | 2.50 |
| Talc | 2.50 | 2.50 | 2.50 |
| Magnesium stearate | 2.50 | 2.50 | 2.50 |

Preparation of Granules Which Constitute the Continued Prolonged/Delayed Release Portion of the Tablets

Granules were prepared from a mixture of ranitidine in the form of ranitidine hydrochloride with Eudragit RS as a binder, used in powder form and/or in the form of an aqueous suspension. Wet granules were dried in a fluid-bed dryer and then milled through a 20 mesh (0.8 mm) screen to obtain appropriate size distribution of the granules suitable for compressing.

Preparation of the Finished Tablets

Granules and the remaining part of the active drug, ranitidine in the form of ranitidine hydrochloride, lipid component, hydrogenated vegetable oil NF, Type I and talc were screened through a 20 mesh sieve and tumble mixed for 5 minutes. Magnesium stearate, screened through a 30 mesh (0.6 mm) sieve was then added to the final blend and mixed for another 5 minutes. The final blend was compressed into tablets.

### Example 9

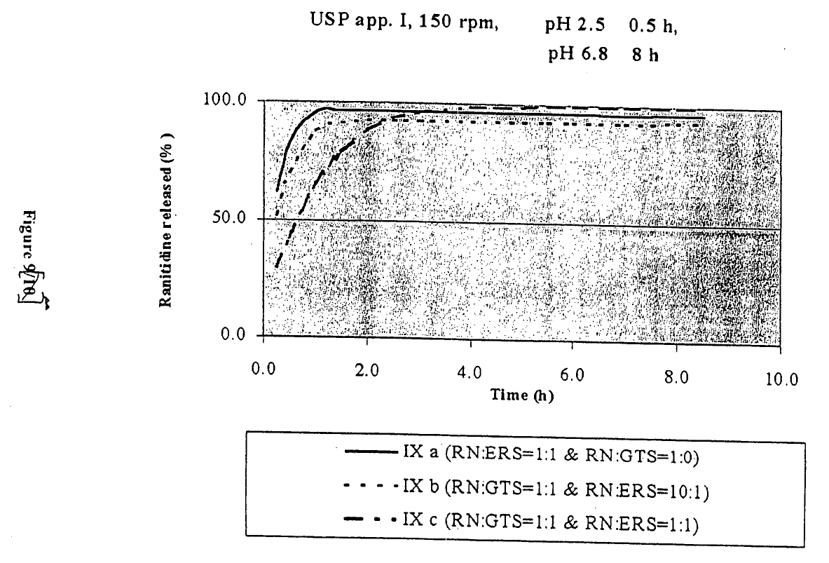

### Controlling the Release Rate by Varying the Amount of Lipid/Lipidic Component as an Additional Retarding Agent (FIG. 9)

| Formulations | IX a | IX b | IX c |
|---|---|---|---|
| GRANULES: Ranitidine:Eudragit RS | 1:1 | 1:1 | 1:1 |
| Ranitidine:glyceril tristearate | without | 10:1 | 1:1 |
| | lipid | | |
| | IX a | IX b | IX c |
| Composition of the tablets | (mg/tbl) | (mg/tbl) | (mg/tbl) |
| Ranitidine (in granules) | 25.00 | 25.00 | 25.00 |
| Eudragit RS | 25.00 | 25.00 | 25.00 |
| Ranitidine (remaining part) | 25.00 | 25.00 | 25.00 |
| Glyceril tristearate | / | 2.50 | 25.00 |
| Hydrogenated vegetable oil NF, Type I | 2.50 | 2.50 | 2.50 |
| Talc | 2.50 | 2.50 | 2.50 |
| Magnesium stearate | 2.50 | 2.50 | 2.50 |

Granules and tablets were prepared in the same way as described in the Example 8.

### Example 10

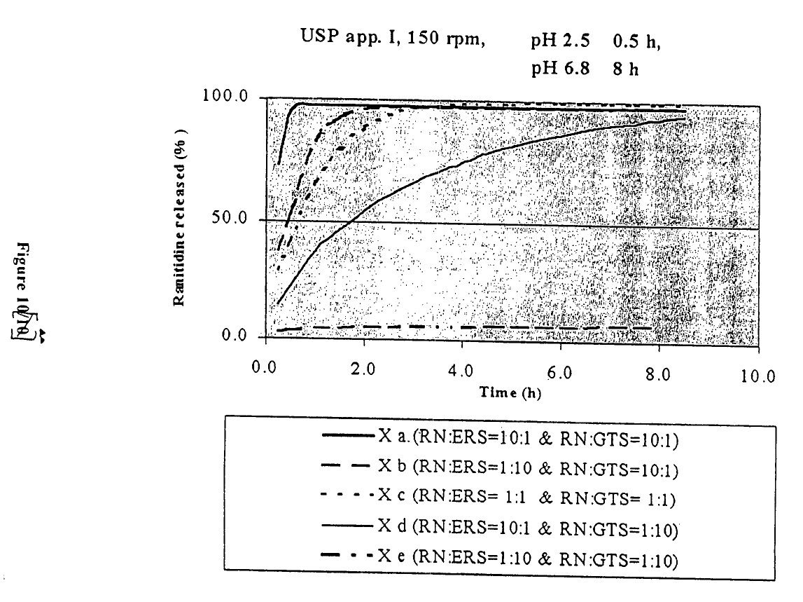

### Controlling the Release Rate by Varying Both the Amount of Water-Insoluble (but Water-Permeable) Polymeric Material and Lipid/Lipidic Component (FIG. 10)

| Formulations | X a | X b | X c | X d | X e |
|---|---|---|---|---|---|
| GRANULES: Ranitidine:Eudragit RS | 10:1 | 1:10 | 1:1 | 10:1 | 1:10 |
| Ranitidine:glyceril tristearate | 10:1 | 10:1 | 1:1 | 1:10 | 1:10 |
| | X a | X b | X c | X d | X e |
| Composition of the tablets | (mg/tbl) | (mg/tbl) | (mg/tbl) | (mg/tbl) | (mg/tbl) |
| Ranitidine (in granules) | 25.00 | 5.00 | 25.00 | 45.00 | 5.00 |
| Eudragit RS | 2.50 | 50.00 | 25.00 | 4.50 | 50.00 |
| Ranitidine (remaining part) | 25.00 | 45.00 | 25.00 | 5.00 | 45.00 |
| Glyceril tristearate | 2.50 | 4.50 | 25.00 | 50.00 | 450.00 |
| Hydrogenated vegetable oil NF, Type I | 2.50 | 2.50 | 2.50 | 2.50 | 2.50 |
| Talc | 2.50 | 2.50 | 2.50 | 2.50 | 2.50 |
| Magnesium stearate | 2.50 | 2.50 | 2.50 | 2.50 | 2.50 |

Granules and tablets were prepared in the same way as described in the Example 8.

It is envisaged that the dosage forms of the present invention will enable controlled delivery of a range of drugs to be provided in a way that maximises therapeutic benefit and patient compliance, while minimising side effects of the drug.

Although the invention has been described with reference to a particular embodiments, it will be appreciated by those people skilled in the art that various alterations and modifications can be made without departing from the spirit and scope of the invention.

1. A sustained-release solid dosage-form, which comprises:
a) granules containing a pharmaceutically active agent intermixed with a pharmaceutically acceptable, water-insoluble, water-permeable polymeric material; and
b) a pharmaceutically active agent and at least 2% (w/w) of a water-insoluble, water-impermeable hydrophobic material.

2. A sustained release solid dosage form as defined in claim 1 a reduced risk of dose dumping and side effects.

3. The pharmaceutical composition as defined in claim 1 wherein the agent in 1b) is not granulated.

4. The pharmaceutical composition as defined in claim 1 wherein the agent in 1b) is in the form of granules to which the water-insoluble, water-impermeable hydrophobic material is added in a melted state.

5. The pharmaceutical composition as defined in claim 1 wherein the agent in 1a) and the agent in 1b) comprise, highly soluble or poorly soluble pharmaceutically active agents, and mixtures of highly soluble and poorly soluble pharmaceutically active agents.

6. The pharmaceutical composition as defined in claim 1 wherein the agent in 1a) and the agent in 1b) are the same active agent.

7. The pharmaceutical composition as defined in claim 1 where the agent in 1a) is different from the agent in 1b).

8. The pharmaceutical composition as defined in claim 6 wherein the active agents are contained in 1a) and 1b) in different amounts.

9. The pharmaceutical composition as defined in claim 1 wherein the water-insoluble, water-permeable polymeric material, in 1a), is selected from the group of one or more of (i) methacrylic acid copolymers or (ii) ethylcellulose or (iii) mixtures thereof.

10. The pharmaceutical composition as defined in claim 9, wherein the water-insoluble, water-permeable polymeric material is in an amount within the range of from about 2 to about 90% (w/w) or in a ratio to the active substance from (1:10) to (10:1), or both.

11. The pharmaceutical composition as defined in claim 1 wherein the water-insoluble, water-impermeable hydrophobic material, in 1b), is selected from the group of hydrogenated vegetable oils and their derivatives, pharmaceutical fats, fatty acids, glycerides and waxes.

12. The pharmaceutical composition as defined in claim 11 wherein the water-insoluble, water-impermeable hydrophobic material in 1b) is in an amount within the range of from about 2 to about 80% (w/w) and/or or in a ratio to the active substance from (1:10) to (10:1), or both.

13. The pharmaceutical composition as defined in claim 1 further comprising one or more fillers in 1a).

14. The pharmaceutical composition as defined in claim 1 further comprising at least one filler or at least one glidant/lubricant or combinations thereof.

15. The pharmaceutical composition as defined in claim 1 further comprising one or more fillers in 1a) and in 1b) at least one filler or at least one glidant/lubricant, or a combination of at least one filler and at least one glidant/lubircant.

16. The pharmaceutical composition as defined in claim 1 wherein a mixture of 1a) and 1b) has been mixed with one or more fillers, one or more glidant/lubricants or a combination of at least one filler and at least one glidant/lubricant.

17. The pharmaceutical composition as defined in claim 5 wherein a mixture of 1a) and 1b) has been compressed into tablets.

18. The pharmaceutical composition as defined in claim 5 wherein a mixture of 1a) and 1b) has been compressed into one or more tablets.

19. The pharmaceutical composition as defined in any of claims 1, 5 and 18 wherein 1a) is compressed into tablets, then mixed with 1b).

20. The pharmaceutical composition as defined in claim 17 the tablets optionally further comprising a film coating comprising one or more film formers, plasticisers and colouring agents.

21. The pharmaceutical composition as defined in claim 20 comprising the following ingredients and amounts:
from about 9 to about 50% (w/w) diclofenac sodium
from about 2 to about 50% (w/w) Eudragit RS
from about 5 to about 35% (w/w) glyceryl tristearate
from about 5 to about 25% (w/w) lactose
from about 10 to about 15% (w/w) microcrystalline cellulose
from about 1 to about 10% (w/w) calcium hydrogen phosphate
from about 1 to about 10% (w/w) hydrogenated vegetable oil NF, type I.

22. The pharmaceutical composition as defined in claim 20 comprising the following ingredients and amounts:
from about 1 to about 85% (w/w) torasemide
from about 2 to about 50% (w/w) Eudragit RS
from about 5 to about 35% (w/w) glyceryl tristearate
from about 5 to about 25% (w/w) lactose
from about 10 to about 15% (w/w) microcrystalline cellulose
from about 1 to about 10% (w/w) calcium hydrogen phosphate
from about 1 to about 10% (w/w) hydrogenated vegetable oil NF, type I.

23. The pharmaceutical composition as defined in claim 20 comprising the following ingredients and amounts:
from about 1 to about 85% (w/w) a ranitidine salt
from about 2 to about 50% (w/w) Eudragit RS
from about 5 to about 35% (w/w) glyceryl tristearate
from about 5 to about 25% (w/w) lactose
from about 10 to about 15% (w/w) microcrystalline-cellulose
from about 1 to about 10% (w/w) calcium hydrogen phosphate
from about 1 to about 10% (w/w) hydrogenated vegetable oil NF, type I.

24. The pharmaceutical composition as defined in claim 1 wherein a mixture of 1a) and 1b) has been filled into hard capsules.

25. The pharmaceutical composition as defined in claim 1 wherein the mixture of 1a) and 1b) has been compressed into one or more tablets and filled into hard capsules.

26. The pharmaceutical composition as defined in claim 1 wherein 1a) has been compressed into tablets, then mixed with the constituents in 1b) and filled into hard capsules.

27. The pharmaceutical composition as defined in claim 1 comprising a suppository comprising a mixture of 1a) and 1b).

28. The pharmaceutical composition as defined in claim 1 wherein a mixture of 1a) and 1b) in the form of a sub-cutaneous implant containing a mixture of 1a) and 1b).

29. A method of preparation of a sustained release solid dosage form as defined in any one of claims 1 and 5 comprising the step of combining:
a first component comprising granules containing a pharmaceutically active agent intermixed with a pharmaceutically acceptable water-insoluble, but water-permeable polymeric material; with
a second component comprising a mixture of a pharmaceutically active agent and at least 2% (w/w) of a water-insoluble, water-impermeable hydrophobic material.

30. A sustained release solid dosage form as defined in any one of claims 1, 5, 17, 18, 19, 21, 22, 23 and 24, adapted for oral administration.

31. A sustained release dosage form as defined in claim 27 adapted for rectal administration.

32. A sustained release dosage form as defined in claim 28 adapted for subcutaneous administration.

33. A method of reducing dose dumping comprising administering to a patient a sustained release solid dosage form as defined in any one of claims 1, 5, 9, 10, 12, 16, 21, 22 and 23.

34. A method for preparing a sustained release dosage form for sustained release of a pharmaceutically active agent comprising combining:
a) granules containing a pharmaceutically active agent intermixed with a pharmaceutically acceptable, water-insoluble, water-permeable polymeric material; and
b) a pharmaceutically active agent and at least 2% (wlw) of a water-insoluble water-impermeable hydrophobic material in a predetermined ratio to yield a dosage form having a desired sustained release profile;

35. A process for the production of a sustained release solid dosage form as defined in claim 1 by combining component a) with component b).

36. The pharmaceutical composition as defined in claim 7 wherein the active agents are contained in 1a) and 1b) in different amounts.

37. The pharmaceutical composition as defined in claim 13 wherein the one or more fillers comprise at least one of lactose and microcrystalline cellulose.

38. The pharmaceutical composition as defined in claim 1 wherein said at least one glidant/lubricant comprises at least one of calcium hydrogen phosphate, hydrogenated vegetable oil NF Type I, talc and/or magnesium stearate.

39. The pharmaceutical composition as defined in claim 18 the tablets optionally further comprising a film coating comprising one or more film formers, plasticisers and colouring agents.

40. The pharmaceutical composition as defined in claim 19 the tablets optionally further comprising a film coating comprising one or more film formers, plasticisers and colouring agents.
